(19) United States
(12) Patent Application Publication
(10) Pub. No.: US 2020/0246148 A1
(43) Pub. Date: Aug. 6, 2020

(54) IMPLANT

(71) Applicant: Friedrich-Alexander Universität Erlangen-Nürnberg, Erlangen (DE)

(72) Inventors: Kolja GELSE, Erlangen (DE); Tobias FEY, Erlangen (DE); Gerhard KRÖNKE, Erlangen (DE); Arnd KLEYER, Erlangen (DE); Axel HUEBER, Erlangen (DE); Nina RENNER, Erlangen (DE); Milena PACHOWSKY, Erlangen (DE); Jonas BIGGEMANN, Erlangen (DE); Carina SCHOLTYSEK, Langenzenn (DE); Peter GREIL, Weisendorf (DE); Georg SCHETT, Erlangen (DE); Marc PEZOLDT, Ilmtal (DE); Martin STUMPF, Erlangen (DE)

(21) Appl. No.: 16/755,914

(22) PCT Filed: Oct. 18, 2018
(86) PCT No.: PCT/EP2018/078555

§ 371 Date: Apr. 14, 2020

(30) Foreign Application Priority Data
Oct. 19, 2017 (DE) 10 2017 124 483.2

(51) Int. Cl. A61F 2/30; A61F 2/28

(52) U.S. Cl. CPC A61F 2/30756; A61F 2/28; A61F 2002/3007; A61F 2002/30321; A61F 2310/00179; A61F 2002/30275; A61F 2002/30433; A61F 2002/30235; A61F 2002/30179; A61F 2002/302; A61F 2002/30266; A61F 2310/00329

(57) ABSTRACT

The invention relates to an implant for replacing bone or cartilage material, which is constituted by a plurality of elements (B, B1, B2, B3, B4) produced from a non-metallic, linearly elastic material, an element (B, B1, B2, B3, B4) being connected to adjacent elements (B, B1, B2, B3, B4) by a viscoelastic polymer material such that gaps (L) remain between the adjacent elements (B, B1, B2, B3, B4) and that the adjacent elements (B, B1, B2, B3, B4) can move relative to one another.

The invention relates to an implant for replacing bone or cartilage material. The implant is particularly suitable for the restoration of functional joint surfaces. The invention additionally relates to a kit.

WO 2014/006519 A1 discloses a three-dimensional porous implant, which is formed from a plurality of stacked layers. The layers are fixedly connected to each other in such a way that gaps or channels remain between adjacent layers. The known implant is particularly unsuitable for the restoration of functional joint surfaces.

Monolithic implants for the restoration of functional joint surfaces are known from the prior art. The following documents are given as examples: DE 103 03 660 B4, EP 0 144 209 B1, EP 0 197 441 B1, DE 100 22 260 02, DE 101 57 315 C1, EP 1 646 334 B1, EP 1 442 726 B1, EP 2 104 471 B1, and EP 2 296 583 B1.

The subsequently published document DE 10 2016 211 201 A1 discloses a flexible bone implant in which structural elements of a first group are connected to structural elements of a second group without gaps. The structural elements have a different hardness.

EP 0 654 250 A discloses a mesh implant for bridging bone defects or for fixing bone fragments.

To restore a functional joint surface by means of a monolithic implant, it is necessary to remove healthy tissue, in particular the biomechanically important subchondral bone plate. This is time-consuming and stressful for the patient. Apart from this, monolithic implants may become loose.

With regard to the restoration of functional joint surfaces, research currently focuses on the repair of cartilage tissue, for example by autologous chondrocyte transplantation, microfracturing or matrix-induced chondrogenesis. This makes it possible to produce cartilage material of sufficient quality. However, it can only be used to treat small chondral lesions with still intact adjacent host cartilage. The treatment of large-area osteoarthritic lesions is therefore not possible.

The object of the invention is to overcome the disadvantages according to the prior art. In particular, an implant shall be described which is easy to apply. According to another aim of the invention, the implant shall be suitable for the restoration of functional joint surfaces and bone material.

This object is achieved by the features of claim 1. Expedient embodiments of the invention will become clear from the features of the dependent claims. A further subject of the invention is a kit.

According to the invention, an implant for replacing bone or cartilage material is proposed, which is constituted by a plurality of elements produced from a non-metallic, linearly elastic material, an element being connected to adjacent elements by a viscoelastic polymer material such that gaps remain between the adjacent elements and that the adjacent elements can move relative to one another.

In the sense of the present invention, the term “gap” is understood to mean a space between a plurality of adjacent elements which are connected to each other by means of the polymer material and which is at least partially delimited by surfaces of the elements. The surfaces may be coated, at least in some sections, with a material which may be different from the polymer material and from the material from which the elements are made. The space may receive a filling material which is different from the polymer material and the material from which the elements are made.

The proposed implant on the whole is a highly flexible structure, in contrast to the prior art. The implant adapts to the shape of a support or cavity due to the viscoelastic properties of the connection of the elements. It is not necessary or is only necessary to a small extent to adapt the support, for example a subchondral bone plate, to the shape of the implant. The connection produced from the viscoelastic material can be easily separated, for example using a knife. As a result, it is quick and easy to adapt the shape of the implant to the shape of the support. The implant can be individually adapted to the conditions of the patient in question.

At least some of the elements made of the non-metallic, linearly elastic material provide a surface that has a high resilience and at the same time low friction. The gaps formed between the elements allow the absorption of cell material, for example chondrogenic or osteogenic cells, or bioactive material after the application of the implant. This enables a cellular colonisation of the implant and thus a durable restoration, for example of a joint surface.

The connection of the elements made of the viscoelastic polymer material enables a largely positive contact of the elements with a support or a cavity. The shape of the implant adapts to the shape of the support or cavity. As a result, valuable, healthy cell material, especially bone tissue, can be largely preserved when using the implant according to the invention.

The proposed implant is constituted from a plurality of elements. The elements may comprise a plurality of elements made of different materials. This makes it possible, for example, to divide the implant into different functional zones. For example, elements made of a non-resorbable ceramic material may be combined with elements made of a resorbable, bioactive ceramic material.

In accordance with a further advantageous embodiment, the linearly elastic material has a modulus of elasticity of at least 10 GPa. The linearly elastic material is substantially brittle. It is usually characterised by a high hardness. The linearly elastic material may also have an open porosity. A communicating pore space usually has a pore volume in the range of 10 to 80 vol. %, preferably 20 to 70 vol. %, particularly preferably 30 to 60 vol. %.

A polymer material with “viscoelastic properties” is understood to be a polymer material which has an elongation at break of at least 0.5%, preferably at least 2%, particularly preferably at least 5%, at room temperature.

Potential linearly elastic materials are metals, non-metallic inorganic materials as well as composites, which may contain polymer materials.

Advantageously, the linearly elastic material is selected from the following group: ceramic, glass ceramic, glass, or a composite material containing at least one of the aforementioned materials. In the composite material, the matrix is advantageously constituted by a polymer material.

The linearly elastic material is selected in particular from the following group: aluminium oxide, hydroxyapatite, beta-tricalcium phosphate (TCP), BaTiO₃ epoxy resin composite, bioglass, bioglass-epoxy resin composites, lead-free epoxy resin composites, lead-free ceramics, e.g. lithium, sodium, potassium-niobate, or ceramic/preceramic polymer composites, for example polysiloxanes, polysilazanes, polyphosphazenes, cross-linked preceramic polymers, and sintered preceramic polymers. The preceramic polymers can be filled with a filler, a proportion of the filler being at least 5 vol. %, preferably at least 20 vol. %, particularly preferably at least 30 vol. %.

In accordance with a further embodiment, at least some of the elements are constituted by a plurality of layers, which are produced from different materials. This makes it possible, for example, to give a surface of the elements facing the bone a function that supports a connection to the bone. A side of the elements facing away from the bone can, for example, be formed from a layer having tribologically advantageous properties. Elements produced from a plurality of layers can be produced, for example, by means of film technology, low-pressure injection moulding, 3D printing, cold/hot compaction techniques and the like.

In accordance with a further embodiment, at least some of the elements comprise an upper and lower side as well as side faces connecting the upper and lower sides. The elements may have a polygonal outline with at least m corners in plan view of the upper side, with m being a natural number ≥3. In particular, the elements can be formed in the manner of a prism or truncated pyramid. It is expedient for the elements to have an n-fold axis of symmetry, with the following being true for n:

n=m/a,

where a is a natural number. This means that the elements can, for example, be a prism with a three- or multi-fold axis of symmetry. Other geometric shapes are also possible, for example an element can be formed in the manner of a cross with eight corners. In this case the cross can have a four-fold axis of symmetry.

Furthermore, at least some of the elements may have an annular or tubular geometry. Such geometries are particularly suitable for the passage of fastening means, such as nails or screws.

Advantageously, a transition between the upper side and the side faces has a rounding. Such a rounding imparts tribologically improved properties to the overall upper side formed by the plurality of elements. Friction at edges in the transition between the upper side and the side faces is avoided.

In accordance with a further embodiment, the upper and/or lower side is curved with a predefined radius. Such a radius can be determined by a radiographic 3D modelling of the bone before the implant is manufactured. This allows an improved, form-fit contact between the implant and the support to be achieved. This means that the predefined radius is adapted to the geometry of the joint.

It is expedient for the upper side to have a first roughness and the lower side to have a second roughness, the first roughness being smaller than the second roughness. For the purposes of the present invention, “roughness” is understood to be the “mean roughness” represented by the symbol Ra. It indicates the average distance of a measuring point on the surface from the centre line. The centre line intersects the actual profile within a reference portion in such a way that the sum of the profile deviations with respect to the centre line is minimal. The average roughness Ra therefore corresponds to the arithmetic mean of the absolute-value deviation from the centre line. It is calculated in two dimensions as follows:

$${R}_{a} = \frac{1}{M N} {\sum }_{m = 1}^{M} {\sum }_{n = 1}^{N} \vert z ( {x}_{m} , {y}_{n} ) - 〈 z 〉 \vert$$

where the mean value is given by

$$〈 z 〉 = \frac{1}{M N} {Σ}_{m = 1}^{M} {Σ}_{n = 1}^{N} z ( {x}_{m} , {y}_{n} ) .$$

In the case of elements with an annular or tubular geometry, a plurality of projections can be moulded on an outer circumference to attach the polymer material. The projections can extend in an axial direction on the outer circumference and have a gable roof-like shape, for example. They can be evenly distributed around the outer circumference. It is advantageous to have at least three such projections on the outer circumference.

In contrast to the linearly elastic material, the polymer material has a modulus of elasticity of less than 10 GPa. The polymer material can be selected in particular from the following group: epoxy resin, preceramic polymers, silicone rubber, collagen, polylactide (PDLA, PLLA), polycaprolactone, polymethylmethacrylates, polylactide-co-glycolide (PLGA), polyhydroxyalkanoates (PHBHHX), fibrin, butyrates, hyaluronic acid, silk, chitosan, alginate. The polymer material may in particular be a biocompatible polymer which is resorbable or non-resorbable.

In accordance with a further advantageous embodiment, the elements comprise a plurality of subsets, with elements of one subset differing from elements of another subset in respect of their geometry. For example, elements with a prismatic geometry can be combined with elements having an annular or tubular geometry.

In accordance with a particularly advantageous embodiment, the viscoelastic polymer material can form a polymer layer which overlays the elements and is attached to the upper side of the elements. The thickness of the polymer layer is expediently in the range of 50 to 1500 μm. The polymer layer may have apertures. It can be reticular or lattice-like.

The proposed polymer layer fulfils advantageously two functions. On the one hand, it serves to flexibly connect the elements. On the other hand, the polymer layer provides a smooth surface, which is in contact with the other half of the joint when the implant is applied. This prevents undesired direct contact of the other half of the joint with corners or edges of the elements. Damage to the other half of the joint can be avoided.—In particular, the polymer layer forms a friction surface having cartilage-like properties. In this way, the surface roughness of the elements can be reduced and/or height differences between the elements can be compensated. Contact between the joint halves can also be avoided by inserting a sliding layer, e.g. a protein layer.

In accordance with a further embodiment, an element is connected to adjacent elements by at least three bridges made of the viscoelastic polymer material. The bridges are connections which are expediently attached to the side faces, in particular to projections or edges of the side faces.

In accordance with a further, particularly advantageous embodiment, the upper side of the elements is coated with the polymer material or a further polymer material. The coating formed from the polymer material or the further polymer material can be connected to the polymer layer at least in some sections.

The implant is particularly suitable for restoring a functional joint surface. Following a further particularly advantageous embodiment, a single layer of elements arranged in one plane is connected by means of the polymer material to form a flexible layer.

In accordance with a further embodiment, a plurality of stacked layers of elements are connected to form a flexible layer or a flexible block. In the embodiment of a flexible layer, the implant is again suitable for the production of functional joint surfaces. In the embodiment of a flexible block, the implant is suitable for filling cavities in bone or generally for modelling bone material. When layers of elements are stacked on top of each other, the elements can be formed, for example, from pyramids with a triangular or polygonal base area. Pyramids formed in the manner of a tetrahedron are particularly suitable for the manufacture of flexible three-dimensional implants. Each pyramidal element is connected via its corners with adjacent pyramidal elements via bridges made of the viscoelastic polymer material.

The invention further relates to a kit comprising an implant according to the invention and fastening means for fastening the implant. The fastening means may be nails, screws and the like, for example. The fastening means may be produced from the material used to manufacture the elements, from metal or from a suitable ceramic. The fastening means are preferably passed through apertures provided in the elements. Annular or tubular elements are particularly suitable for the passage of the fastening means.

In the following, exemplary embodiments of the invention are explained in more detail using the drawings, in which:

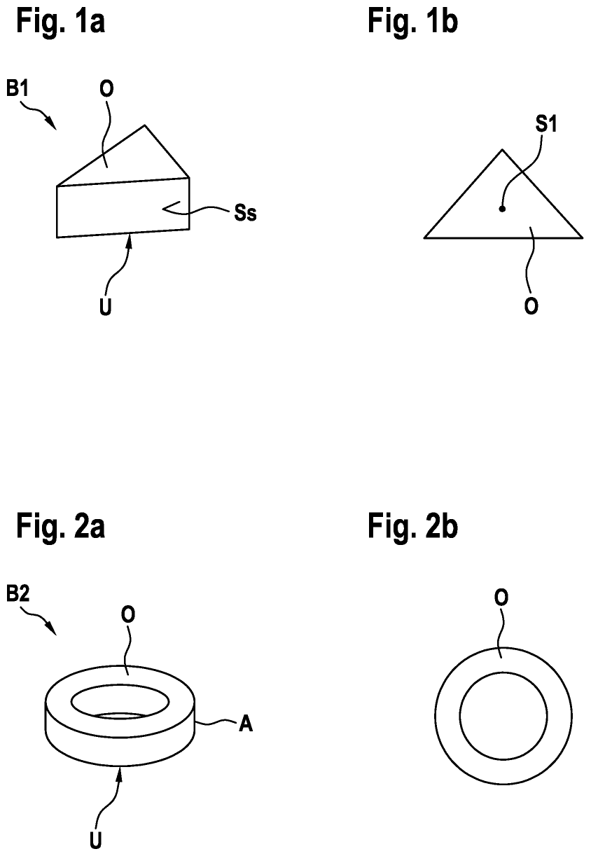

FIG. 1a shows a perspective view of a first element;

FIG. 1b shows a plan view of the element according to FIG. 1a;

FIG. 2a shows a perspective view of a second element;

FIG. 2b shows a plan view of the element according to FIG. 2a;

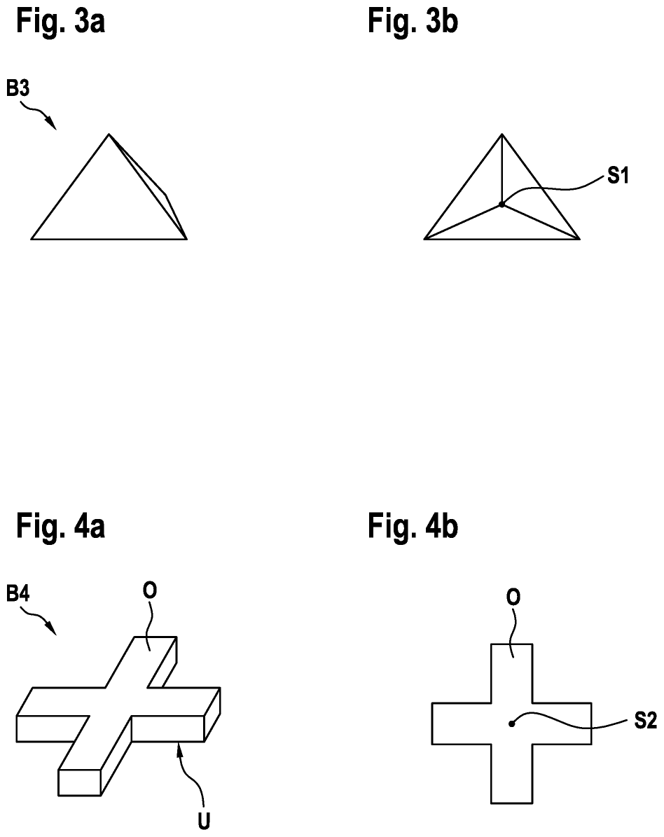

FIG. 3a shows a perspective view of a third element;

FIG. 3b shows a plan view of the element according to FIG. 3a;

FIG. 4a shows a perspective view of a fourth element;

FIG. 4b shows a plan view of the element according to FIG. 4a;

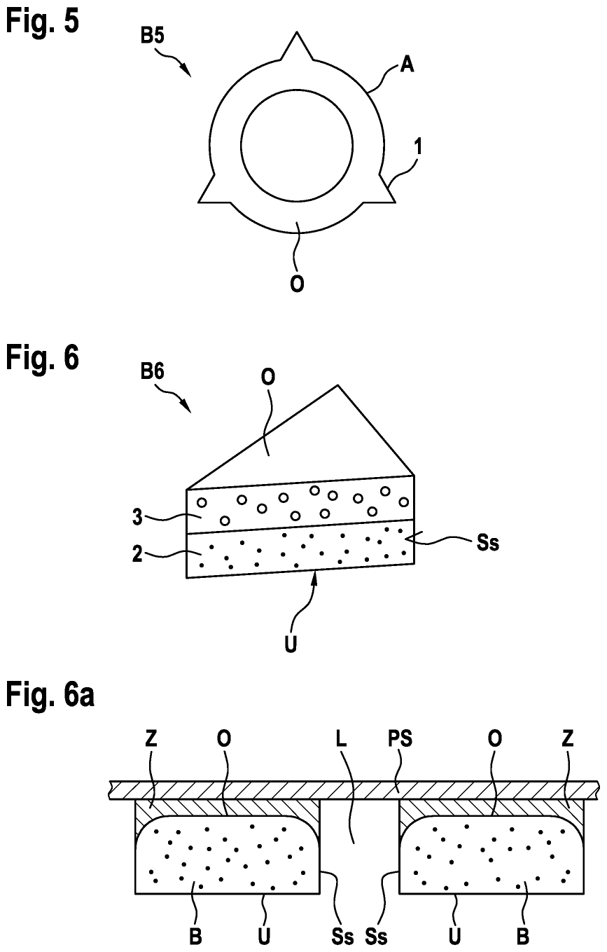

FIG. 5 shows a plan view of a variant of the second element;

FIG. 6 shows a perspective view of a variant of the first element;

FIG. 6a shows a schematic view of two elements with a gap between them;

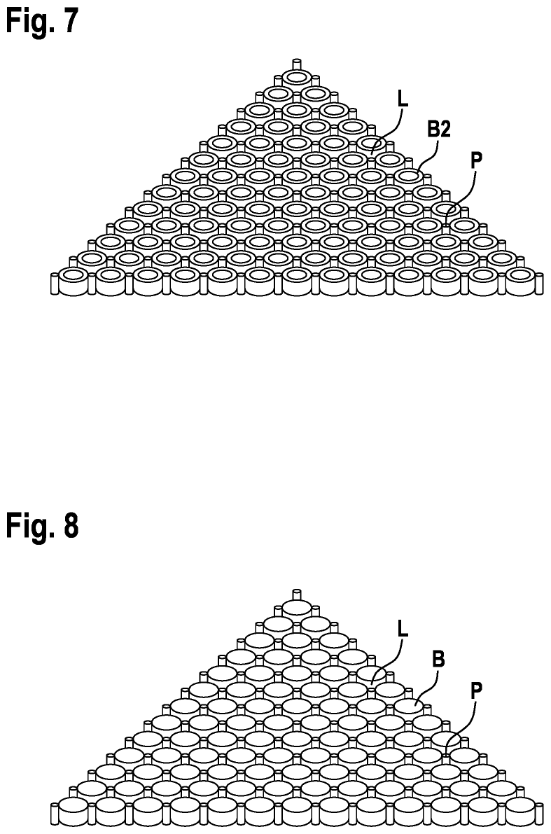

FIG. 7 shows a perspective view of a first implant,

FIG. 8 shows a perspective view of a second implant;

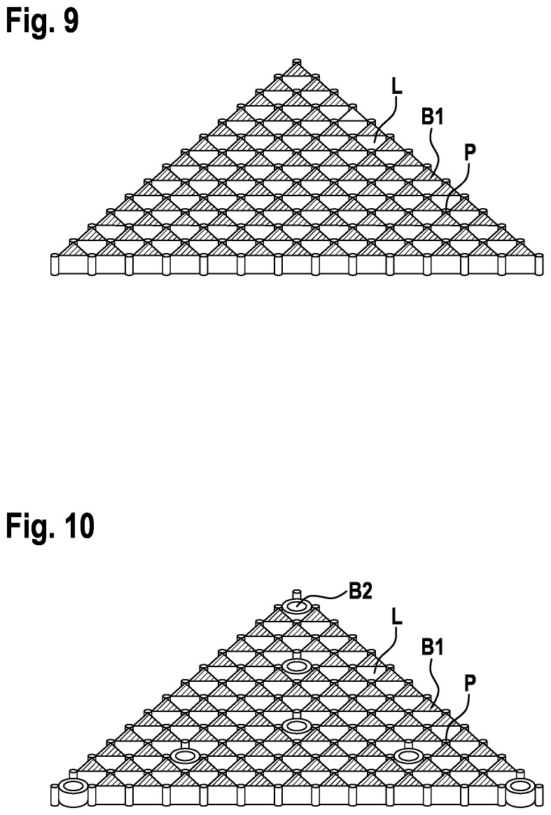

FIG. 9 shows a perspective view of a third implant;

FIG. 10 shows a perspective view of a fourth implant;

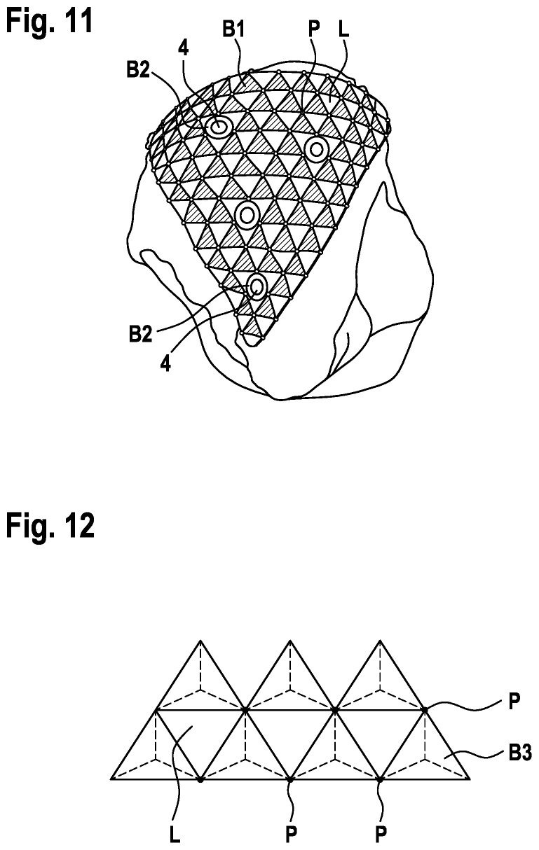

FIG. 11 shows a perspective view of the fourth implant in a form applied to a bone;

FIG. 12 shows a schematic view of a fifth implant;

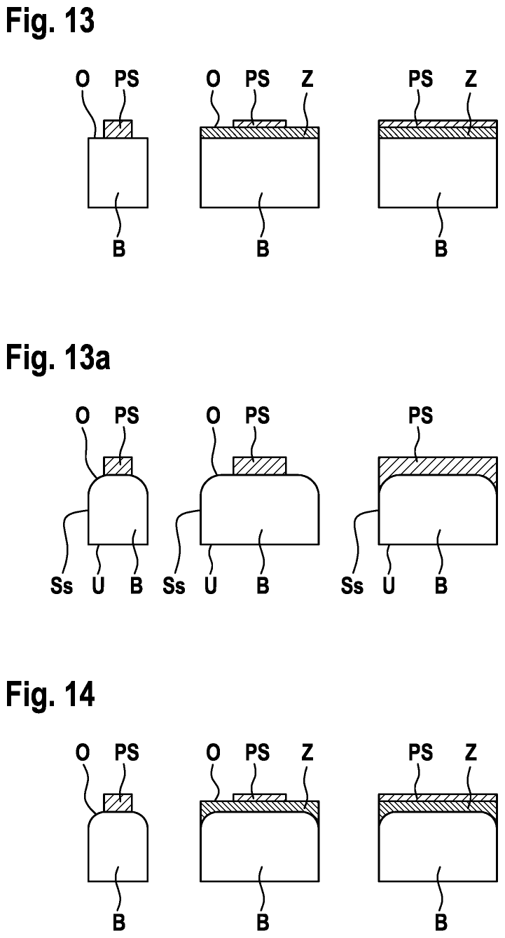

FIG. 13 shows a schematic view of elements with a polymer layer attached to them;

FIG. 13a shows a schematic view of further elements with a coating;

FIG. 14 shows a schematic view of further elements with intermediate and polymer layers attached to them;

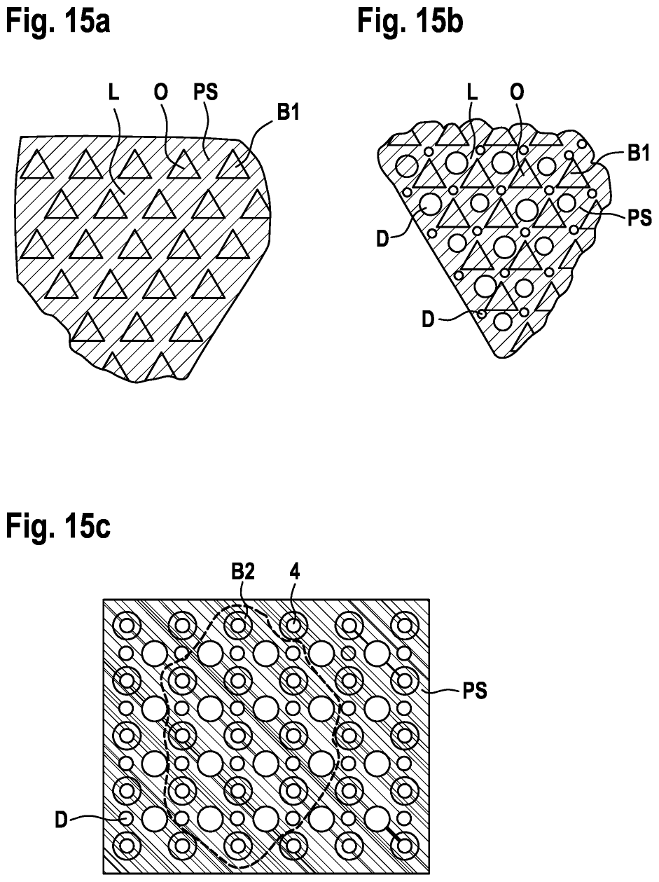

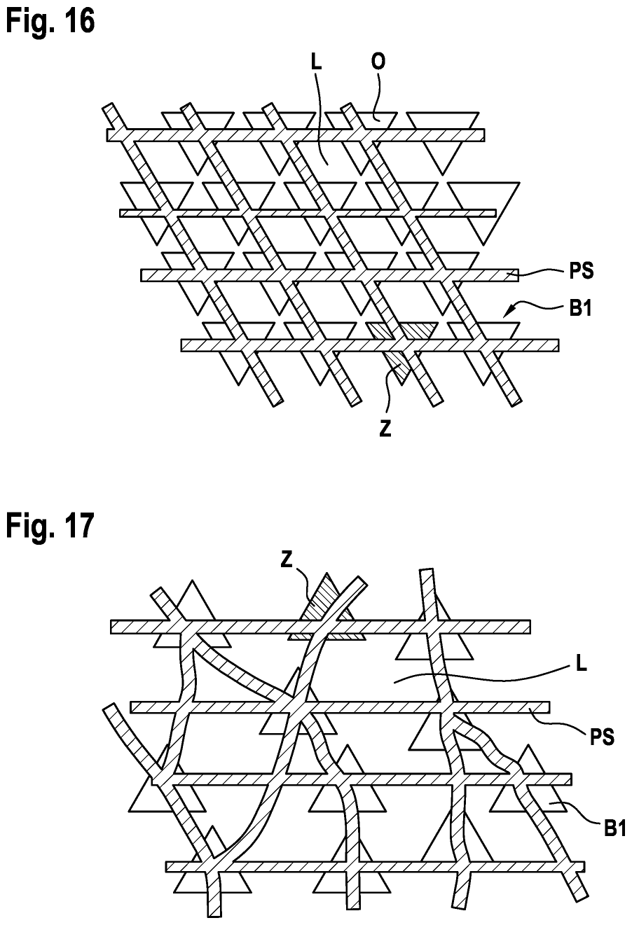

FIG. 15a shows a schematic view of a sixth implant;

FIG. 15b shows a schematic view of a seventh implant;

FIG. 15c shows a schematic view of an eighth implant;

FIG. 16 shows a schematic plan view of a ninth implant;

FIG. 17 shows a schematic plan view of a tenth implant; and

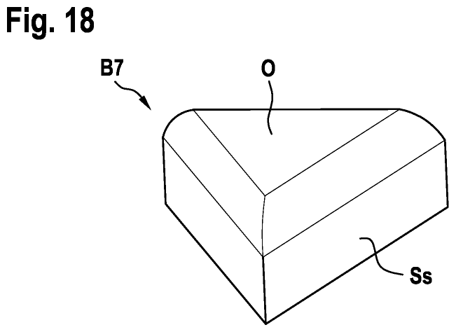

FIG. 18 shows a perspective view of a seventh element.

FIGS. 1 to 6 and 18 show examples of elements B from which implants according to the invention can be produced. A maximum diameter of the elements B can be 0.3 to 10.0 mm, preferably 0.5 to 7.0 mm, particularly preferably 2.0 to 6.0 mm. For the production of a first variant of an implant, which is shown in FIGS. 7 to 12 as an example, the elements B are connected by means of flexible bridges P to form a flexible structure. In the second variant shown in FIGS. 14 to 17, the elements B are connected to form a flexible structure by means of a polymer layer, which may be in the form of a polymer lattice.

The following tables give examples of suitable materials for the production of the elements B as well as suitable polymer materials:

TABLE 1
Material for elements

| | Modulus of | | |
|---|---|---|---|
| | elasticity | KIC | |
| Material | [GPa] | [MPa/m^{1/2}] | Behaviour |
| Aluminium oxide Al₂O₃ | 385 | 3.6-4.4 | Linear-elastic |
| Hydroxyapatite | 80-120 | 0.6-1.0 | Linear-elastic |
| Beta-TCP | 21 | 2.3 | Linear-elastic |
| Bioglass | 35 | 2 | Linear-elastic |
| Porous Al₂O₃ | 60-200 | | Linear-elastic |
| (30-70% porosity) | | | |
| BaTiO₃ - | 12-30 | | Linear-elastic |
| Epoxy resin composites | | | |
| (5-45 vol. % BaTiO3) | | | |
| LNKN - | 12-30 | | Linear-elastic |
| Epoxy resin composites | | | |
| (5-45 vol. % LNKN) | | | |
| Cortical bone | 7-30 | 2-12 | |

TABLE 2
Polymer materials

| | Modulus of | | Elongation | |
|---|---|---|---|---|
| | elasticity | KIC [MPa/ | at break | |
| Material | [GPa] | m^{1/2}] | [%] | Behaviour |
| Epoxy resin | 4-8 | | 0.5-6, | Visco- |
| | | | Epicure 9.4 | elastic |
| Silicone rubber | 0.045 | 0.03 | 2-100 | Visco- |
| | | | | elastic |
| Collagen | 0.3-2.5 | 1-10 | 10-30 | Visco- |
| | | | | elastic |
| Polylactides | 2.3-3.5 | | 2-6, 5.3 | Visco- |
| | | | | elastic |
| Poly- | | | 1-200, | Visco- |
| caprolactones | | | partly 660 | elastic |
| Polymethyl- | 1.8-3.3 | | 1-100, | Visco- |
| methacrylates | | | Vitralit 4731 | elastic |
| | | | (328) | |
| Poly (lactic-co- | | | 2-8 | Visco- |
| gly-colic acid) | | | | elastic |
| (PLGA) | | | | |
| Polyhydroxyalka- | | | 8-15 | Visco- |
| noates (hexano- | | | | elastic |
| ates) (PHBHHx) | | | | |
| Fibrin | | | | |
| Butyrates | | | | |
| Hyaluronic acid | 0.1-0.4 | | | |
| Silk | 0.01-0.4 | | | |
| Chitosan | 0.8-1.2 | | | |
| Alginate | 14 kPa | | | |

FIGS. 1a and 1b show a first element B1, which is designed in the manner of a trigonal prism. An upper side of the first element B1 is marked with the reference sign O, a lower side with the reference sign U and the side faces connecting the upper side O to the lower side U are denoted by the reference sign Ss. The base area of the first element B1 is formed by an equilateral triangle. The first element B1 has a three-fold first axis of symmetry S1.

FIGS. 2a and 2b show a second element B2. The second element B2 is formed in the manner of a pipe portion. An outer circumference is denoted by the reference sign A.

FIGS. 3a and 3b show a third element B3, which is shaped like a tetrahedron. The base area of the tetrahedron is an equilateral triangle in a three-fold first axis of symmetry S1.

FIGS. 4a and 4b show a fourth element B4. The fourth element B4 is shaped like a cross. It has eight corners and a four-fold second axis of symmetry S2 in the plan view.

FIG. 5 shows a variant of the second element B2 as the fifth element B5. In this case, projections 1 are moulded on an outer circumference A. The projections 1 are evenly distributed over the outer circumference A.

FIG. 6 shows a variant of the first element B1 as the sixth element B6. The sixth element B6 is formed from a first layer 2 and a second layer 3 thereabove. The first layer 2, which comprises the lower side U, is for example produced from a material that supports an attachment to a support, for example bone or cartilage tissue. The second layer 3 is produced from a different material. This can be a tribologically resilient material, for example aluminium oxide, a bioactive material or the like. The second layer 3 can also be formed from a sequence of a plurality of second layers. The layers can be formed from the linear-elastic material and/or the viscoelastic material or a combination of both materials.—The upper side O may be provided with a coating Z, which is made of the polymer material and/or a further polymer material which is different from the polymer material used (not shown here).

FIG. 6a shows a schematic sectional view through two elements B with a gap L in between. The gap L is bounded by the side faces Ss of the elements B. In the embodiment shown in FIG. 6a, the edges delimiting the upper side O of the elements B are rounded. The coating Z is provided on the upper side O as well as a portion of the side faces Ss. In the embodiment shown here, the gaps L are therefore also delimited in some sections by the coating Z. It is also possible that the side faces Ss are completely covered with the coating Z. In this case, the gaps L are limited by the side faces Ss provided with the coating Z. A filling material (not shown here) may be included in the gap L. The filler material may be constituted by cells, a cell-matrix construct, bioactive material, e.g. encapsulated cells, growth and/or differentiation factors or the like.

FIGS. 7 to 10 show, for example, implants for the restoration of functional joint surfaces. The implants shown are each formed from elements B arranged in a single plane, which are flexibly connected to each other by means of a plurality of bridges P. Gaps between the elements B are denoted by the reference sign L. When applied, the gaps L serve to accommodate cell material or bioactive material.

For the first implant shown in FIG. 7, the elements B correspond to the second element B2 shown in FIGS. 2a and 2b. Each element B is connected to adjacent elements B via three bridges P.

In the second implant shown in FIG. 8, the elements B are cylindrical. Here, too, each element B is connected to adjacent elements B via three bridges P.

In the third implant shown in FIG. 9, the elements B are formed according to the first element B1 shown in FIGS. 1a and 1b. Here, too, the adjacent elements B are each connected to one another by three bridges P. The connections or bridges P are each attached to the edges of elements B running approximately perpendicular to the upper side O.

The fourth implant shown in FIG. 10 combines elements B of different geometries. The fourth implant comprises first elements B1 and second elements B2. The first elements B1 and the second elements B2 are again flexibly connected to one another in each case by three bridges P. The second elements B2 are used for the passage of fastening means, for example screws or nails 4.

In the exemplary embodiments shown in FIGS. 7 to 10, the bridges P have a geometrically defined, specifically cylindrical, shape.—However, the connections or bridges P can also be geometrically different from one another in respect of their shape.

FIG. 11 shows the fourth implant according to FIG. 10 in a form applied to a bone. Fastening means, e.g. nails 4, pass through the second elements B2 and anchor the fourth implant in the bone. It is also possible that a pin intended for fastening purposes is connected in one piece to an element and extends for example from its underside.

FIG. 12 schematically shows a fifth implant, which is formed from layers placed one on top of the other. Each layer is formed from third elements B3 flexibly connected to one another by means of bridges P. The layers are again connected to one another by means of bridges P. Instead of the third elements formed from tetrahedra, bipyramidal elements can also be used in a similar way, for example trigonal or tetragonal bipyramidal elements.

FIG. 13 schematically shows elements B, with a polymer layer PS attached to the upper side O. The upper side O can be provided with a coating Z, which is produced from the polymer material and/or another polymer material which is different from the polymer material used. In this case the coating Z is located between the element and the polymer layer PS. The further polymer material may be selected from the materials specified for the polymer material. The polymer layer PS can cover the upper side O in sections or over the entire surface.

FIG. 13a schematically shows elements B, whose edges adjacent to the upper side O are rounded.

FIG. 14 shows further elements B, to the upper sides of which the polymer layer PS is attached. The edges of the further elements B adjacent to the upper side O are rounded here. The coating Z provided on the upper side O can cover the upper side O in sections or over the entire surface. The coating Z can also extend beyond the rounded edges to the side faces. It can also cover the side faces at least in some sections.

FIG. 15a shows a schematic view of a sixth implant. In the sixth implant, the polymer layer PS is continuous, i.e. without apertures. It is attached to the upper side of the first elements B1. The first elements B1 are arranged regularly.

In the seventh implant shown in FIG. 15b, the polymer layer PS has regularly arranged apertures D. The apertures D are located above the gaps L between the first elements B1.

FIG. 15c schematically shows an eighth implant. The eighth implant is formed by second elements B2, which are preferably arranged regularly. The second elements B2 can be rounded at their upper side O. The polymer layer PS is attached to the upper side O or an intermediate layer Z applied to the upper side O (not shown here). The polymer layer PS has round apertures, preferably arranged regularly.—The eighth implant shown can form a semi-finished product. The surgeon can cut a suitable piece out of the eighth implant as required. A corresponding cutting line is shown as an example by the interrupted line. The second elements B2 are universally suitable for the passage of fastening means such as nails 4, screws and the like. Because of the use of the second elements B2, the surgeon has a lot of freedom with regard to the application of fastening means.

In the ninth implant shown schematically in FIG. 16, the first elements B1 are also arranged regularly. The polymer layer PS here consists of a regular grid. As can be seen in FIG. 16, the surfaces O of the first elements B1 can be completely or only partially overlaid with the polymer layer PS. Some of the first B1 elements may be provided with the coating Z.

In the tenth implant shown in FIG. 17, the first elements B1 are irregularly arranged. The polymer layer PS is formed from an irregularly shaped grid. Some of the first elements B1 may be provided with the coating Z.

FIG. 18 shows a perspective view of a seventh element B7. The seventh element B7 is similar to the first element B1, but here the edges adjacent to the upper side O are rounded.

The polymer layer PS can be present as a polymer film. The polymer layer PS can be connected to coated or uncoated elements B by means of a thermally activated pressing process. The polymer layer PS, however, can also be produced by 3D direct printing, for example using the FDM process, on coated or uncoated elements B. Irregularly shaped grids in particular allow the implant to be adjusted to different degrees of flexibility. However, the flexibility can also be varied by varying the thickness of the polymer layer PS and/or the size of the connection area between the elements B and the polymer layer PS and/or the size and number of the apertures.

Especially in implants formed from a single layer of elements B, the gaps L form cavities in the state applied to a support. Such cavities can be filled with a cell-loaded or cell-free matrix. The matrix may contain growth and differentiation factors that promote cell migration and/or chondrogenic or osteogenic differentiation of the cells.

The cavities can be filled with a cell-matrix construct. Such a cell-matrix construct comprises autologous and/or allogeneic mesenchymal stem and/or progenitor cells or autologous chondrocytes or periosteum cells. The cells can be applied in a biocompatible matrix, for example collagen, hyaluronic acid, alginate, chitosan, fibrin or in biopolymers. A cell-free matrix can also be applied into the cavities. In this case, the cells can be integrated into the matrix via connections to the bone marrow space, for example by drilling holes or subchondral bone lamellae.

In accordance with a further embodiment the implant can also be prepared with gaps already filled. The filling material may include cells, a cell-matrix construct, growth and/or differentiation factors and the like. In particular, the cells, cell-matrix constructs and the like mentioned in the previous two paragraphs can be used as filling material.

### LIST OF REFERENCE SIGNS

1 projection
2 first layer
3 second layer
4 nail
B element
B1 first element
B2 second element
B3 third element
B4 fourth element
B5 fifth element
B6 sixth element
B7 seventh element
D aperture
L gap
O upper side
P bridge
PS polymer layer
S1 first axis of symmetry
S2 second axis of symmetry
Ss side face
U lower side
Z coating

1. An implant for replacing bone or cartilage material, which is constituted by a plurality of elements produced from a non-metallic, linearly elastic material, an element being connected to adjacent elements by a viscoelastic polymer material such that gaps remain between the adjacent elements and that the adjacent elements can move relative to one another, the gaps being used to accommodate cell material or bioactive material in the applied state,
wherein the polymer material is selected from the following group: epoxy resin, preceramic polymers, silicone rubber, polylactide, polycaprolactone, polymethylmethacrylates, polylactide-co-glycolide (PLGA), polyhydroxyalkanoates (PHBHHX), fibrin, butyrates, silk, chitosan,
wherein the polymer material forms a polymer layer which overlays the elements and is attached to the upper side of the elements, and
wherein the polymer layer has apertures.

2. The implant according to claim 1, wherein the elements comprise a plurality of elements produced from different materials.

3. The implant according to claim 1, wherein the linearly elastic material has a modulus of elasticity of at least 10 GPa.

4. The implant according to claim 1, wherein the linearly elastic material is selected from the group consisting of: ceramic, glass ceramic, glass, or a composite material containing at least one of the aforementioned materials.

5. The implant according to claim 1, wherein the linearly elastic material is selected from the group consisting of: aluminium oxide, hydroxyapatite, beta-tricalcium phosphate (TCP), BaTiO₃ epoxy resin composite, bioglass, bioglass epoxy resin composites, lead-free epoxy resin composites, lead-free ceramics, lithium, sodium, potassium niobate, ceramic/preceramic polymer composites, polysiloxanes, polysilazanes, polyphosphazenes, cross-linked preceramic polymers, and sintered preceramic polymers.

6. The implant according to claim 1, wherein at least some of the elements are produced from a plurality of layers made of a different material.

7. The implant according to claim 1, wherein at least some of the elements comprise an upper side and lower side as well as side faces connecting the upper side to the lower side, the elements having a polygonal outline with at least m corners in plan view of the upper side, where m is a natural number ≥3.

8. The implant according to claim 1, wherein at least some of the elements have an n-fold axis of symmetry, with the following being true for n:
n=m/a,
where a is a natural number.

9. The implant according to claim 1, wherein at least some of the elements have an annular or tubular geometry.

10. The implant according to claim 1, wherein a transition between the upper side and the side faces has a rounded shape.

11. The implant according to claim 1, wherein the upper side and/or lower side is curved with a predefined radius.

12. The implant according to claim 1, wherein the upper side has a first roughness and the lower side has a second roughness, the first roughness being smaller than the second roughness.

13. The implant according to claim 1, wherein a plurality of projections for attaching the polymer material are formed on an outer circumference.

14. The implant according to claim 1, wherein the polymer material has a modulus of elasticity of less than 10 GPa.

15. (canceled)

16. The implant according to claim 1, wherein the elements comprise a plurality of subsets, with elements of one subset differing from the elements of another subset in respect of their geometry.

17. (canceled)

18. The implant according to claim 1, wherein the polymer layer has a thickness in the range of 50 to 1500 μm.

19. (canceled)

20. The implant according to claim 1, wherein the polymer layer is reticular or lattice-like.

21. The implant according to claim 1, wherein an element is connected to adjacent elements by at least three bridges made of the viscoelastic polymer material.

22. The implant according to claim 1, wherein the upper side and/or side face of the elements is coated with the polymer material and/or a further polymer material.

23. The implant according to claim 1, wherein a single layer of elements arranged in one plane are joined together by means of the polymer material to form a flexible layer.

24. The implant according to claim 1, wherein a plurality of stacked layers of elements are connected to form a flexible layer or a flexible block.

25. A kit comprising an implant according to claim 1 and fastening means for fastening the implant.
